(19) United States
(12) Patent Application Publication
(10) Pub. No.: US 2026/0102719 A1
(43) Pub. Date: Apr. 16, 2026

(54) DUAL-SPECIFIC BINDING MOLECULE USEFUL FOR ISOLATING SPECIFIC BLOOD CELLS

(71) Applicant: ImmunityBio, Inc., San Diego, CA (US)

(72) Inventors: Patrick SOON-SHIONG, El Segundo, CA (US); Paul WEINGARTEN, El Segundo, CA (US)

(73) Assignee: ImmunityBio, Inc., San Diego, CA (US)

(21) Appl. No.: 19/357,552

(22) Filed: Oct. 14, 2025

Related U.S. Application Data
(63) Continuation of application No. PCT/US2025/050786, filed on Oct. 14, 2025.
(60) Provisional application No. 63/707,463, filed on Oct. 15, 2024.

(51) Int. Cl. B01D 21/01; B01D 21/26; C07K 16/28; C12N 9/64

(52) U.S. Cl. CPC B01D 21/01; B01D 21/262; C07K 16/2896; C12N 9/6489; C12Y 304/24087; C07K 2319/33

(57) ABSTRACT

Provided herein are methods and compositions for the removal and isolation of target blood cells from a whole blood sample using bifunctional fusion proteins. The fusion proteins may include a first binding domain that is specific for a red blood cell (RBC) surface antigen, a cleavable or stable linker, and a second binding domain that is specific for a cell-surface expressed antigen of a target blood cell. The methods may involve adding the fusion protein to the whole blood, followed by centrifugation to produce a pellet comprising RBCs bound to target blood cells. The target blood cells may include non-RBC cell types such as antigen-presenting cells, which may express an MHC-presented peptide derived from an infectious agent or cancer-associated antigen. The target blood cells may be further separated from RBCs by lysing or cleaving the linker. Also provided are compositions, kits, and methods for identifying infected or cancerous white blood cells using MHC-targeted binding domains.

### CROSS REFERENCE TO RELATED APPLICATIONS

This application is a continuation of PCT/US25/50786 filed on Oct. 14, 2025, which claims the benefit of U.S. Provisional Application No. 63/707,463, filed Oct. 15, 2024, the entire contents of each of which are herein incorporated by reference in their entireties for all purposes.

### FIELD

The present disclosure concerns bispecific binding molecules and uses thereof. The binding molecules disclosed herein are useful for isolating specific cells (e.g., natural killer cells, T or B cells, and infectious microorganisms) from a sample of whole blood.

### BACKGROUND

US 2025/0051473 (IGM Biosciences, Inc.) describes a bispecific binding molecule that can bind to a cell-surface antigen on erythrocytes and to a cell-surface antigen on T cells.

U.S. Pat. No. 6,887,377 (Sigma-Aldrich Co.) describes immunoprecipitation of specific target molecules using microbeads centrifuged out of suspension.

Current methods for isolating specific cell types from whole blood often require multi-step processing, harsh chemical treatments, or expensive equipment, which can compromise cell viability and reduce efficiency. In particular, selectively removing and identifying rare or disease-associated immune cells remains technically challenging, especially when such cells must be distinguished based on surface markers or presented peptide antigens. Conventional approaches may also lack the precision to isolate specific subpopulations without significant loss or contamination from other blood components. These limitations hinder both diagnostic accuracy and therapeutic cell preparation. Accordingly, a need exists for improved methods and compositions for the selective removal, identification, and isolation of target blood cells directly from whole blood.

### SUMMARY

The present disclosure provides compositions, including bispecific binding molecules, and methods thereof for isolating specific cells (e.g., natural killer cells, T or B cells, and infectious microorganisms) from a sample of whole blood.

In some aspects, the present disclosure provides a method for removing and isolating cells from a whole blood sample. In some aspects, the method may include adding to the whole blood sample a fusion protein including a first binding domain, a linker, and a second binding domain. In some aspects, the first binding domain may be specific for a cell-surface expressed antigen of a red blood cell (RBC). In some aspects, the second binding domain may be specific for a cell-surface expressed antigen of a target blood cell. In some aspects, the method may include centrifuging the whole blood to produce a pellet including target-cell bound RBC. In some aspects, the RBC and target cells may be removed from the blood cells remaining in the supernatant. In some aspects, the target cells may be isolated in the RBC pellet.

In some aspects, the target blood cell is not an RBC. In some aspects, the target blood cell may be selected from the group including a natural killer (NK) cell, a T cell, an αβT cell, a γδT cell, an invariant NKT cell, a dendritic cell, a B cell, a granulocyte, a platelet, and a macrophage. In some aspects, the RBC antigen may be selected from the group including glycophorin A (CD235), glycophorin B, glycophorin C, solute carrier family 4A1 (SLC4A1), and SLC2A1. In some aspects, the RBC antigen may be glycophorin A.

In some aspects, the cell-surface expressed antigen of a target blood cell may include an MHC-I or MHC-II presented peptide. In some aspects, the method may be used for the removal of target blood cells from the whole blood, wherein the target blood cells may include antigen presenting cells. In some aspects, the antigen presenting cells may be infected by a virus. In some aspects, the antigen presenting cells may include a cancer-inducing genetic mutation. In some aspects, the second binding domain may include a single chain T cell receptor (scTCR).

In some aspects, the linker may be stable. In some aspects, the linker may be cleavable. In some aspects, the target cells may be further separated from the red blood cells. In some aspects, the red blood cells may be lysed under hypotonic conditions. In some aspects, the pellet may be resuspended, the linker may be cleaved, and the suspension including RBC and target cells may be recentrifuged, whereby the RBC may be pelleted.

In some aspects, the present disclosure provides a composition including a whole blood sample and a fusion protein. In some aspects, the fusion protein may include a first binding domain, a linker, and a second binding domain. In some aspects, the first binding domain may be specific for a cell-surface expressed antigen of an RBC. In some aspects, the second binding domain may be specific for a cell-surface expressed antigen of a target blood cell.

In some aspects, the first binding domain may include the antigen binding domain of an antibody or antibody-like fragment, or a single chain fragment variable (scFv). In some aspects, the second binding domain may include the antigen binding domain of an antibody or antibody-like fragment, or an scFv. In some aspects, the second binding domain may be specific for a cell-surface expressed antigen of a target blood cell selected from the group including a natural killer (NK) cell, a T cell, an αβT cell, a γδT cell, an invariant NKT cell, a dendritic cell, a B cell, a granulocyte, a platelet, and a macrophage. In some aspects, the second binding domain may include an scTCR. In some aspects, the linker may be cleavable. In some aspects, the first binding domain may include the 175-kD erythrocyte binding antigen (EBA-175) of Plasmodium falciparum.

In some aspects, the present disclosure provides a method for identifying an infected white blood cell in a whole blood sample. In some aspects, the method may include adding to the whole blood sample a fusion protein including a first binding domain, a linker, and a second binding domain. In some aspects, the first binding domain may be specific for a cell-surface expressed antigen of an RBC. In some aspects, the second binding domain may be specific for an MHC-complexed peptide antigen of a target blood cell. In some aspects, the peptide antigen may be derived from an infectious agent. In some aspects, the method may include centrifuging the whole blood to produce a pellet including infected target cells and RBC.

In some aspects, the present disclosure provides a method for identifying a cancerous white blood cell in a whole blood sample. In some aspects, the method may include adding to the whole blood sample a fusion protein including a first binding domain, a linker, and a second binding domain. In some aspects, the first binding domain may be specific for a cell-surface expressed antigen of an RBC. In some aspects, the second binding domain may be specific for an MHC-complexed peptide antigen of a target blood cell. In some aspects, the peptide antigen may be derived from a tumor specific antigen, a tumor associated antigen, or a neoantigen. In some aspects, the method may include centrifuging the whole blood to produce a pellet including cancerous target cells and RBC.

In some aspects, the present disclosure provides a kit including a whole blood sample and a fusion protein. In some aspects, the fusion protein may include a first binding domain, a linker, and a second binding domain. In some aspects, the first binding domain may be specific for a cell-surface expressed antigen of an RBC. In some aspects, the second binding domain may be specific for a cell-surface expressed antigen of a target blood cell.

In some aspects, the present disclosure provides a composition including a whole blood sample, a first fusion protein, and a second fusion protein. In some aspects, the first fusion protein may include a first binding domain, an optional linker, and a second binding domain. In some aspects, the first binding domain may be specific for a first cell-surface expressed antigen of an RBC. In some aspects, the second binding domain may be specific for a cell-surface expressed antigen of a first target blood cell. In some aspects, the second fusion protein may include a third binding domain, an optional linker, and a fourth binding domain. In some aspects, the third binding domain may be specific for a second cell-surface expressed antigen of an RBC. In some aspects, the fourth binding domain may be specific for a cell-surface expressed antigen of a second target blood cell.

In some aspects, the first and second cell-surface expressed antigens of the RBC may be different. In some aspects, the second and fourth binding domains may be specific for a cell-surface expressed antigen of a target blood cell selected from the group including a natural killer (NK) cell, a T cell, an αβT cell, a γδT cell, an invariant NKT cell, a dendritic cell, a B cell, a granulocyte, a platelet, and a macrophage. In some aspects, the target cell targeted by the second binding domain may be different than the target cell targeted by the fourth binding domain. In some aspects, the RBC antigen may be selected from the list including CD235, glycophorin B, glycophorin C, SLC4A1, and SLC2A1. In some aspects, the first and third RBC cell-surface expressed antigens may be different.

In some aspects, the present disclosure provides a composition including a whole blood sample, a first fusion protein, and a second fusion protein. In some aspects, the first fusion protein may include a first binding domain, an optional linker, and a second binding domain. In some aspects, the first and second binding domains may be specific for a first cell-surface expressed antigen of an RBC. In some aspects, the second fusion protein may include a third binding domain, an optional linker, and a fourth binding domain. In some aspects, the third binding domain may be specific for a second cell-surface expressed antigen of an RBC. In some aspects, the fourth binding domain may be specific for a cell-surface expressed antigen of a target blood cell.

### BRIEF DESCRIPTION OF THE DRAWINGS

FIG. 1 is a schematic showing a bispecific binding molecule with a binding moiety (circle) specific for a T cell surface antigen and a binding moiety (diamond) specific for an RBC surface antigen, joined by a linker (extended black line between circle and diamond). The RBC-binding end is shown bound to an RBC, while the T cell-binding end is shown bound to a T cell.

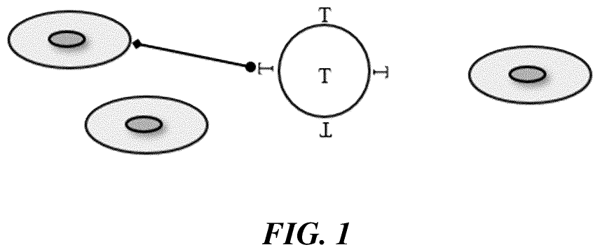

### DETAILED DESCRIPTION

The present disclosure describes compositions, including bispecific binding molecules, and methods thereof for isolating specific cells (e.g., natural killer cells, T or B cells, and infectious microorganisms) from a sample of whole blood. The compositions and methods described herein may be used to identify or remove immune cell subtypes associated with infection or disease. Various aspects may be practiced without some or all of these specific details. In certain instances, well-known operations have been described in less detail so as not to obscure the embodiments.

As used herein, “fusion protein” refers to a polypeptide that includes at least two distinct binding domains connected by a linker sequence. In some aspects, the fusion protein may include an N-terminal binding domain. In some aspects, the fusion protein may include a C-terminal binding domain. In some aspects, the fusion protein may include both an N-terminal binding domain and a C-terminal binding domain.

As used herein, a “binding domain” refers to any protein domain or polypeptide motif that specifically binds to a target antigen, including but not limited to antibody fragments, T cell receptor fragments, or pathogen-derived ligands.

As used herein, a “linker” refers to any amino acid sequence that connects two protein domains. In some aspects, the linker may be stable. In some aspects, the linker may be cleavable.

As used herein, “cleavable” refers to linkers that may be severed under defined chemical, enzymatic, or physical conditions, such as by proteases, pH shifts, or reducing agents.

As used herein, “target blood cell” refers to any non-RBC cell found in whole blood, including immune cells, platelets, or rare circulating cells.

As used herein, “MHC-complexed peptide antigen” refers to any antigenic peptide that is presented at the cell surface by major histocompatibility complex (MHC) molecules.

### Methods

Methods of removing and isolating cells from a whole blood sample are disclosed herein. The method includes adding a fusion protein to the whole blood sample. The fusion protein may include a first binding domain, a linker, and a second binding domain. The first binding domain may be specific for an RBC antigen, and the second binding domain may be specific for a target blood cell antigen.

In some aspects, the method may include centrifuging the whole blood to produce a pellet including red blood cells (RBCs) bound to target cells. In this way, the RBCs and target cells may be removed from other cells present in the supernatant, and/or the target cells may be isolated within the RBC pellet.

In some aspects, the target blood cell is an RBC, while in other aspects the target blood cell will not be an RBC. By way of non-limiting example, the target blood cell may be selected from the group including natural killer (NK) cells, T cells, αβT cells, γδT cells, invariant NKT cells, dendritic cells, B cells, granulocytes, platelets, and/or macrophages.

In order to capture an RBC from the whole blood sample, the RBC-binding domain should be specific for an antigen found on the surface of an RBC, such as (but not limited to) an antigen selected from CD235 (glycophorin A), glycophorin B, glycophorin C, solute carrier family 4A1 (SLC4A1), and SLC2A1.

In some aspects, the disclosed methods are used for the treatment of B cell-mediated autoimmune diseases. The B cell-mediated autoimmune diseases may be immune Thrombotic Thrombocytopenic Purpura (iTTP), systemic lupus erythematosus, myasthenia gravis, or autoimmune hemolytic anemia. In some aspects, the second binding domain of the fusion protein may be comprised of an autoantigen targeted by pathogenic B cells. By way of non-limiting example, in the treatment of iTTP, the second binding domain may be comprised of ADAMTS13 or an antibody-binding domain thereof, a plasma metalloprotease deficient in functional activity due to pathogenic anti-ADAMTS13 antibodies. Binding of the fusion protein to ADAMTS13-specific B cells in patient blood enables selective removal of these B cells following incubation and centrifugation. In some aspects, an optional pre-treatment step to remove or reduce free antigen-specific immunoglobulins from the patient sample (e.g., IgG purification) is performed prior to fusion protein addition, thereby reducing competition between soluble antibodies and the target B cell receptors for antigen binding.

In some aspects, the target blood cell antigen will be an MHC-I-complexed peptide and/or MHC-II-complexed peptide, i.e. an MHC-I complexed peptide. Where this complexed peptide relates to an infectious agent or a tumor, the method disclosed herein can be used for the removal of target blood cells that include antigen-presenting cells.

By way of non-limiting example, the antigen-presenting cells may be infected by a non-viral pathogen, such as a bacterium, parasite, fungus, prion-associated particle, and/or synthetic or engineered microbe. Additionally or alternatively, the antigen-presenting cells may be infected by a virus, such as a human immunodeficiency virus (HIV) or a severe acute respiratory syndrome coronavirus (e.g., SARS-CoV2). Additionally or alternatively, the antigen-presenting cells may include a cancer-inducing genetic mutation. Additionally or alternatively, the antigen-presenting cells may include a non-cancer-inducing genetic mutation.

The second binding domain of the fusion protein may include a single chain T cell receptor (scTCR). For example, where the target antigen is an MHC-complexed peptide, the scTCR may be engineered to bind specific MHC-presented peptides.

While the fusion protein may directly connect the first and second binding domains, it is also possible for the first and second binding domains to be connected by a linker. The linker between binding domains may be stable. However, the linker between binding domains may be stable only in specific environments, or for a specific period of time. For example, the linker may be cleavable (e.g., when in the presence of one or more matrix metalloproteases). Additionally or alternatively, the linker may not be cleavable.

In the course of the methods disclosed herein, the target cells may be further separated from red blood cells. For example, the RBCs may be lysed under hypotonic conditions, and/or the pellet resuspended, the linker cleaved, and the suspension recentrifuged, such that free RBCs are pelleted and target cells recovered from the supernatant or interphase.

The present disclosure also provides methods for identifying an infected or cancerous white blood cell in a whole blood sample. These methods include adding to the sample a fusion protein including a first binding domain specific for an RBC antigen and a second binding domain specific for an MHC-complexed peptide antigen on the white blood cell.

By way of non-limiting example, the MHC-complexed peptide antigen may be derived from an infectious agent, a tumor-specific antigen, a tumor-associated antigen, or a neoantigen. Centrifugation of the sample produces a pellet including the target white blood cell and RBC. The pellet may then include a target white blood cell which is infected and/or cancerous.

In some aspects, the method further includes adding—in combination with a fusion protein having an RBC-binding domain on one end and a target cell-binding domain on the other end—a second fusion protein having RBC-binding domains on both ends. The dual-RBC-binding fusion protein may promote aggregation or cross-linking of RBCs, thereby facilitating more efficient pelleting of RBCs and associated target cells during centrifugation. In some aspects, both RBC-binding domains of the dual-RBC-binding fusion protein are specific for the same RBC antigen, while in other aspects the domains are specific for different RBC antigens, which may enhance RBC aggregation and stability of the RBC-target cell complex.

### Compositions

The present disclosure also provides compositions including a whole blood sample and a fusion protein. As explained earlier, the fusion protein include a first binding domain specific for a cell-surface expressed antigen of an RBC and a second binding domain specific for a cell-surface expressed antigen of a target blood cell, optionally connected by a linker. The arrangement of these binding domains is not critical. The first binding domain may be a C-terminal domain, joined to the N-terminal second domain, or the first binding domain may be an N-terminal domain, joined to the C-terminal second domain. In either arrangement, the first may be joined directly to the second, or a linker may interpose between the first and second binding domains.

The first binding domain may include the antigen-binding domain of an antibody, an antibody-like fragment, or a single-chain variable fragment (scFv). Additionally or alternatively, the first binding domain may include the EBA-175 of P. falciparum, which is known to bind glycophorin A on RBCs. Additionally or alternatively, the second binding domain may independently include an antibody fragment, an scFv, or other targeting moiety.

The second binding domain may be specific for a target blood cell selected from the group including NK cells, T cells, αβT cells, γδT cells, invariant NKT cells, dendritic cells, B cells, granulocytes, platelets, and macrophages.

The second binding domain may include an scTCR. In some aspects, the scTCR may recognize an MHC-complexed peptide antigen associated with disease or infection.

Where desired, the linker connecting the first and second binding domains may be cleavable. Additionally or alternatively, the linker connecting the first and second binding domains may not be cleavable. A cleavable linker may enable dissociation of the RBC-cell complex under specific conditions, such as enzymatic treatment or chemical cleavage.

Additionally or alternatively, the composition may include more than one fusion protein. For example, a composition may include both a first fusion protein with a first binding domain specific for a first RBC antigen and a second binding domain specific for a first target blood cell, and a second fusion protein with a first binding domain specific for a second RBC antigen and a second binding domain specific for a second target blood cell. One or both of these fusion proteins may also include a linker. In some aspects, the composition includes a second fusion protein having an RBC-binding domain on both the N-terminal and C-terminal ends. Such a dual-RBC-binding fusion protein may be co-added with a fusion protein having an RBC-binding domain on one end and a target cell-binding domain on the other end. The presence of the dual-RBC-binding fusion protein can promote aggregation or cross-linking of RBCs, thereby increasing the efficiency of RBC-associated pull-down of the target cells during centrifugation. In some aspects, the dual-RBC-binding fusion protein binds the same RBC antigen on both ends, while in other aspects, each end binds a different RBC antigen, potentially enhancing the stability and avidity of RBC aggregation.

In some aspects, the composition is formulated for use in the treatment of B cell-mediated autoimmune diseases, such as iTTP, systemic lupus erythematosus, myasthenia gravis, or autoimmune hemolytic anemia. For example, in the treatment of iTTP, the second binding domain of the RBC-target cell fusion protein may be comprised of ADAMTS13, enabling selective binding to and removal of ADAMTS13-specific B cells from patient blood. The dual-RBC-binding fusion protein may be included to enhance the pelleting efficiency of these bound B cells during centrifugation, thereby improving B cell depletion.

The first and second RBC antigens may be the same. Additionally or alternatively, the first and second RBC antigens may be different. The target cells bound by the second and fourth binding domains may be the same. Additionally or alternatively, the target cells bound by the second and fourth binding domains may be different. The target blood cells may be selected from the group including NK cells, T cells, αβT cells, γδT cells, invariant NKT cells, dendritic cells, B cells, granulocytes, platelets, and macrophages.

In some aspects, the RBC-binding domains of the first and second fusion proteins may target RBC antigens selected from the group including CD235 (glycophorin A), glycophorin B, glycophorin C, solute carrier family 4A1 (SLC4A1), and SLC2A1. For example, the first and second RBC-binding domains may be specific for different antigens within this list.

### Kits

The present disclosure provides a kit including a whole blood sample and a fusion protein. The fusion protein will be as described above, i.e., it will include a first binding domain (e.g., a binding domain specific for an RBC antigen) and a second binding domain (e.g., a binding domain specific for a target blood cell antigen). Additionally or alternatively, the fusion protein may include a linker (e.g., cleavable linker or non-cleavable linker).

Additional aspects are described below.

Aspect 1. A method of removing and isolating cells from a whole blood sample, the method comprising: 1) adding to the whole blood sample a fusion protein comprising a first binding domain, a linker, and a second binding domain, wherein the first binding domain is specific for a cell-surface expressed antigen of a red blood cell (RBC) and wherein the second binding domain is specific for a cell-surface expressed antigen of a target blood cell; 2) centrifuging the whole blood to produce a pellet comprising target-cell bound RBC; wherein the RBC and target cells have been removed from the blood cells remaining in the supernatant, and wherein the target cells are isolated in the RBC pellet.

Aspect 2. The method of aspect 1, wherein the target blood cell is not an RBC.

Aspect 3. The method of aspect 1 or 2, wherein the target blood cell is selected from the list comprising a natural killer (NK) cell, a T cell, an αβT cell, a γδT cell, an invariant NKT cell, a dendritic cell, a B cell, a granulocyte, a platelet, and a macrophage.

Aspect 4. The method of any one of the previous aspects, wherein the RBC antigen is selected from the group consisting of glycophorin A (CD235), glycophorin B, glycophorin C, solute carrier family 4A1 (SLC4A1), and SLC2A1.

Aspect 5. The method of aspect 4, wherein the RBC antigen is glycophorin A (CD235).

Aspect 6. The method of any one of the previous aspects, wherein the cell-surface expressed antigen of a target blood cell comprises an MHC-I or MHC-II presented peptide.

Aspect 7. The method of aspect 6, for use in the removal of target blood cells from the whole blood, wherein the target blood cells comprise antigen presenting cells.

Aspect 8. The method of aspect 7, wherein the antigen presenting cells may be infected by a virus.

Aspect 9. The method of aspect 7 or 8, wherein the antigen presenting cells comprise a cancer-inducing genetic mutation.

Aspect 10. The method of any one of the previous aspects, wherein the second binding domain comprises a single chain T cell receptor (scTCR).

Aspect 11. The method of any one of the previous aspects, wherein the linker is stable.

Aspect 12. The method of any one of the previous aspects, wherein the linker is cleavable.

Aspect 13. The method of any one of the previous aspects, wherein the target cells are further separated from the red blood cells.

Aspect 14. The method of aspect 13, wherein the red blood cells are lysed under hypotonic conditions.

Aspect 15. The method of aspect 13 or 14, wherein the pellet is resuspended, the linker is cleaved, and the suspension comprising RBC and target cells is recentrifuged, whereby the RBC are pelleted.

Aspect 16. A composition comprising a whole blood sample and a fusion protein, wherein the fusion protein comprises a first binding domain, a linker, and a second binding domain, wherein the first binding domain is specific for a cell-surface expressed antigen of an RBC and wherein the second binding domain is specific for a cell-surface expressed antigen of a target blood cell.

Aspect 17. The composition of aspect 16, wherein the first binding domain comprises the antigen binding domain of an antibody or antibody-like fragment, or a single chain fragment variable (scFv).

Aspect 18. The composition of aspect 16 or 17, wherein the second binding domain comprises the antigen binding domain of an antibody or antibody-like fragment, or an scFv.

Aspect 19. The composition of any one of aspects 16 to 18, wherein the second binding domain is specific for a cell-surface expressed antigen of a target blood cell selected from the group consisting of a natural killer (NK) cell, a T cell, an αβT cell, a γδT cell, an invariant NKT cell, a dendritic cell, a B cell, a granulocyte, a platelet, and a macrophage.

Aspect 20. The composition of any one of aspects 16 to 19, wherein the second binding domain comprises an scTCR.

Aspect 21. The composition of any one of aspects 16 to 20, wherein the linker is cleavable.

Aspect 22. The composition of any one of aspects 16 to 21, wherein the first binding domain comprises the 175-kD erythrocyte binding antigen (EBA-175) of Plasmodium falciparum.

Aspect 23. A method identifying an infected white blood cell in a whole blood sample, the method comprising: 1) adding to the whole blood sample a fusion protein comprising a first binding domain, a linker, and a second binding domain, wherein the first binding domain is specific for a cell-surface expressed antigen of an RBC and wherein the second binding domain is specific for an MHC-complexed peptide antigen of a target blood cell, wherein the peptide antigen is derived from an infectious agent; 2) centrifuging the whole blood to produce a pellet comprising infected target cells and RBC.

Aspect 24. A method identifying a cancerous white blood cell in a whole blood sample, the method comprising: 1) adding to the whole blood sample a fusion protein comprising a first binding domain, a linker, and a second binding domain, wherein the first binding domain is specific for a cell-surface expressed antigen of an RBC and wherein the second binding domain is specific for an MHC-complexed peptide antigen of a target blood cell, wherein the peptide antigen is derived from a tumor specific antigen, a tumor associated antigen, or a neoantigen; 2) centrifuging the whole blood to produce a pellet comprising cancerous target cells and RBC.

Aspect 25. A kit comprising a whole blood sample and a fusion protein, wherein the fusion protein comprises a first binding domain, a linker, and a second binding domain, wherein the first binding domain is specific for a cell-surface expressed antigen of an RBC and wherein the second binding domain is specific for a cell-surface expressed antigen of a target blood cell.

Aspect 26. A composition comprising a whole blood sample, a first fusion protein, and a second fusion protein, wherein the first fusion protein comprises a first binding domain, an optional linker, and a second binding domain, wherein the first binding domain is specific for a first cell-surface expressed antigen of an RBC and wherein the second binding domain is specific for a cell-surface expressed antigen of a first target blood cell, and wherein the second fusion protein comprises a third binding domain, an optional linker, and a fourth binding domain, wherein the third binding domain is specific for a second cell-surface expressed antigen of an RBC and wherein the fourth binding domain is specific for a cell-surface expressed antigen of a second target blood cell.

Aspect 27. The composition of aspect 26, wherein the first and second cell-surface expressed antigens of the RBC are different.

Aspect 28. The composition of aspect 26 or 27, wherein the second and fourth binding domains are specific for a cell-surface expressed antigen of a target blood cell selected from the group consisting of a natural killer (NK) cell, a T cell, an αβT cell, a γδT cell, an invariant NKT cell, a dendritic cell, a B cell, a granulocyte, a platelet, and a macrophage, and wherein the target cell targeted by the second binding domain is different than the target cell targeted by the fourth binding domain.

Aspect 29. The composition of any one of aspects 26 to 28, wherein the RBC antigen is selected from the list consisting of CD235, glycophorin B, glycophorin C, SLC4A1, and SLC2A1, and wherein the first and third RBC cell-surface expressed antigens are different.

Aspect 30. A composition comprising a whole blood sample, a first fusion protein, and a second fusion protein, wherein the first fusion protein comprises a first binding domain, an optional linker, and a second binding domain, wherein the first and second binding domains are specific for a first cell-surface expressed antigen of an RBC, and wherein the second fusion protein comprises a third binding domain, an optional linker, and a fourth binding domain, wherein the third binding domain is specific for a second cell-surface expressed antigen of an RBC and wherein the fourth binding domain is specific for a cell-surface expressed antigen of a target blood cell.

### EXAMPLES

While various aspects have been described herein, they have been presented by way of example only, and not limitation. Thus, the breadth and scope of the present disclosure should not be limited by any of the described exemplary aspects. Moreover, any combination of the above-described elements in all possible variations thereof is encompassed by the disclosure unless otherwise indicated herein or otherwise clearly contradicted by context.

### Example 1

A fusion protein will be prepared that includes (1) a first binding domain specific for glycophorin A (CD235) expressed on red blood cells (RBCs), (2) a cleavable linker, and (3) a second binding domain comprising a single chain T cell receptor (scTCR) specific for a class I MHC peptide presented on dendritic cells infected with Epstein-Barr Virus (EBV).

The first binding domain will include a single-chain fragment variable (scFv) derived from an anti-CD235 antibody (e.g., ab129024 from abCM, Waltham, Massachusetts). The cleavable linker will comprise a protease-cleavable peptide sequence that is stable in blood but cleavable upon exposure to matrix metalloproteinase-9 (MMP-9). The second binding domain will include an scTCR engineered to recognize the YVL-BR epitope of EBV presented on HLA-A*02: 01.

The fusion protein will be produced recombinantly in CHO cells and purified by affinity chromatography followed by size exclusion filtration. Following purification, the fusion protein will be added to freshly collected human whole blood (collected in heparinized tubes) at a final concentration of 1 μg/mL. The blood will be incubated with the fusion protein at 4° C. for 15 minutes to allow binding. The sample will then be centrifuged at 500 g for 5 minutes at 4° C. The resulting pellet will include RBCs crosslinked to EBV-infected dendritic cells. The supernatant will be aspirated and discarded.

The pellet will be resuspended in hypotonic buffer to lyse RBCs, and then centrifuged again to isolate the target dendritic cells. Alternatively, the cleavable linker will be enzymatically cleaved by addition of activated MMP-9 to release the target cells for downstream analysis.

### Example 2

A sample of human whole blood will be obtained and maintained at 4° C. A fusion protein will be added to the sample at a final concentration of 1 μg/mL. The fusion protein will include a first binding domain specific for a red blood cell (RBC) surface antigen (e.g., CD235), a linker susceptible to degradation at pH<5, and a second binding domain specific for a target blood cell antigen. The second binding domain will comprise a single chain T cell receptor (scTCR) specific for an MHC-I or MHC-II complexed peptide antigen.

Following addition of the fusion protein, the blood sample will be incubated for 10 minutes at 4° C. to allow binding of the fusion protein to both RBCs and target blood cells. The sample will then be centrifuged at 500 g for 5 minutes at 4° C. The resulting pellet will include RBCs bound to target blood cells.

The supernatant will be removed. The pellet will be resuspended in hypotonic lysis buffer to lyse the RBCs and release the target blood cells. Alternatively, the pellet will be treated with acid to sever the linker, followed by recentrifugation to separate free target cells from RBCs.

Recovered target cells may be resuspended in buffer and analyzed using flow cytometry, PCR-based methods, or other downstream analytical techniques.

### Example 3

A fusion protein will be prepared that includes (1) a first binding domain specific for glycophorin A expressed on RBCs; and (2) a second binding domain comprising full-length ADAMTS13 protein. The first binding domain will include a single-chain fragment variable (scFv) derived from an anti-CD235 antibody. The first and second binding domains will be joined by a flexible, non-cleavable linker.

Patient whole blood will be collected during a standard apheresis procedure from a patient diagnosed with iTTP. Prior to addition of the fusion protein, the apheresis product may be subjected to immunoglobulin depletion using protein A or protein G affinity resin to remove circulating anti-ADAMTS13 antibodies. Alternatively, the plasma may be set aside and the cells resuspended in immunoglobulin-free and ADAMTS13-free medium.

The fusion protein will be added to the patient's leukocyte-containing fraction and incubated to allow binding of ADAMTS13 to B cell receptors specific for ADAMTS13. The sample will then be centrifuged to produce a pellet containing RBCs bound to ADAMTS13-specific B cells. The supernatant will be aspirated and discarded. The pellet will then be separated from other cell types by lysing RBCs under hypotonic conditions or by density-gradient centrifugation.

Following removal of ADAMTS13-specific B cells, donor RBCs will be added back to the processed blood product to restore red cell content, and the processed leukocyte-reduced plasma will be reinfused into the patient. This process may be repeated as necessary to further deplete pathogenic B cells while allowing restoration of functional ADAMTS13 activity via standard replacement therapy.

The foregoing description has been presented for purposes of illustration and description. This description is not intended to limit the invention to the precise form disclosed. Persons of ordinary skill in the art will appreciate that modifications and substitutions of the basic inventive description may be made. All patents and published applications referenced in this disclosure are herein incorporated by reference in their entireties.

What is claimed is:

1. A method of removing and isolating cells from a whole blood sample, the method comprising 1) adding to the whole blood sample a fusion protein comprising a first binding domain, a linker, and a second binding domain, wherein the first binding domain is specific for a cell-surface expressed antigen of a red blood cell (RBC) and wherein the second binding domain is specific for a cell-surface expressed antigen of a target blood cell; 2) centrifuging the whole blood to produce a pellet comprising target-cell bound RBC; wherein the RBC and target cells have been removed from the blood cells remaining in the supernatant, and wherein the target cells are isolated in the RBC pellet.

2. The method of claim 1, wherein the target blood cell is not an RBC.

3. The method of claim 1, wherein the target blood cell is selected from the list comprising a natural killer (NK) cell, a T cell, an abT cell, a gdT cell, an invariant NKT cell, a dendritic cell, a B cell, a granulocyte, a platelet, and a macrophage.

4. The method of claim 1, wherein the RBC antigen is selected from the group consisting of glycophorin A (CD235), glycophorin B, glycophorin C, solute carrier family 4A1 (SLC4A1) and SLC2A1.

5. The method of claim 4, wherein the RBC antigen is glycophorin A (CD235).

6. The method of claim 4, wherein the cell-surface expressed antigen of a target blood cell comprises an MHC-I or MHC-II presented peptide.

7. The method of claim 6, wherein target blood cells are removed from the whole blood, and wherein the target blood cells comprise antigen presenting cells (APCs).

8. The method of claim 7, wherein the APCs are infected by a virus.

9. The method of claim 7, wherein the APCs comprise a cancer-inducing genetic mutation.

10. The method of claim 6, wherein the second binding domain comprises a single chain T cell receptor (scTCR).

11. The method of claim 6, wherein the linker is stable.

12. The method of claim 6, wherein the linker is cleavable.

13. The method of claim 7, wherein the target cells are further separated from the red blood cells.

14. The method of claim 13, wherein the red blood cells are lysed under hypotonic conditions.

15. The method of claim 13, wherein pellet is resuspended, the linker is cleaved, and the suspension comprising RBC and target cells is recentrifuged, whereby the RBC are pelleted.

16. A composition comprising a whole blood sample and a fusion protein, wherein the fusion protein comprises a first binding domain, a linker, and a second binding domain, wherein the first binding domain is specific for a cell-surface expressed antigen of an RBC and wherein the second binding domain is specific for a cell-surface expressed antigen of a target blood cell.

17. The composition of claim 16, wherein the first binding domain comprises the antigen binding domain of an antibody or antibody-like fragment, or a single chain fragment variable (scFv).

18. The composition of claim 16, wherein the second binding domain comprises the antigen binding domain of an antibody or antibody-like fragment, or an scFv.

19. The composition of claim 18, wherein the second binding domain is specific for a cell-surface expressed antigen of a target blood cell selected from the group consisting of a natural killer (NK) cell, a T cell, an abT cell, a gdT cell, an invariant NKT cell, a dendritic cell, a B cell, a granulocyte, a platelet, and a macrophage.

20. The composition of claim 16, wherein the second binding domain comprises an scTCR.

21. The composition of claim 16, wherein the linker is cleavable.

22. The composition of claim 16, wherein the first binding domain comprises the 175-kD erythrocyte binding antigen (EBA-175) of Plasmodium falciparum.

23. A method of treating immune Thrombotic Thrombocytopenic Purpura (iTTP) in a patient in need thereof, the method comprising: (a) incubating a fusion protein comprising (1) a first binding domain specific for glycophorin A expressed on RBCs and (2) a second binding domain comprising an ADAMTS13 protein or an antibody-binding domain thereof in a blood sample from the patient; (b) centrifuging the blood sample to create a supernatant and a pellet containing anti-ADAMTS13 auto-antibodies; and (c) re-infusing the supernatant into the patient.

24. The method of claim 23, wherein the first binding domain comprises an scFv derived from an anti-CD235 antibody.

25. The method of claim 23, wherein the first and the second binding domains are joined by a flexible, non-cleavable linker.

26. The method of claim 23, further comprising incubating the blood sample with protein A or protein G prior to incubating with the fusion protein.

27. The method of claim 23, further comprising adding donor RBCs to the supernatant to restore RBC content, prior to the reinfusing.
